(19) United States
(12) Patent Application Publication
(10) Pub. No.: US 2007/0123828 A1
(43) Pub. Date: May 31, 2007

(54) PIV hub cushion kit

(75) Inventor: Donald J. Propp, Dewitt, MI (US)

Correspondence Address: Christopher J. Fildes;Fildes & Outland, P.C., Suite 2, 20916 Mack Avenue, Grosse Pointe Woods, MI, 48236, US

(73) Assignee: Tri-State Hospital Supply Corporation

(21) Appl. No.: 11/289,917

(22) Filed: Nov. 30, 2005

(51) Int. Cl. A61M 5/32

(52) U.S. Cl. 604/180; 604/174

(57) ABSTRACT

A catheter patient mounting accessory kit includes a release liner having a support side. The kit also includes at least one accessory member having an adhesive side and an opposite non-adhesive side adapted for use in combination with a medical dressing. Additionally, the kit includes at least one other accessory member having an adhesive side and an opposite non-adhesive side, and being adapted for use as a means for securement. Each of the accessory member adhesive sides are releasably disposed on the release liner support side.

### TECHNICAL FIELD

This invention relates to indwelling catheters, and more specifically to a catheter patient mounting accessory kit to facilitate convenient, comfortable mounting of the catheter to a patient.

### BACKGROUND OF THE INVENTION

It is known in the medical and surgical fields to administer intravenous (IV) fluids to a patient through a catheter inserted into a vein of the patient. The catheter is typically coupled to an IV connector and/or IV extension set, which is connected by IV tubing to a container of IV fluid. Generally, the catheter includes a connecting hub which cooperates with an IV connector for connecting the catheter to upstream medical connectors and/or tubing.

Once the catheter is inserted into the patient, it is desirable to stabilize the catheter to prevent a rocking or teeter totter movement and to make the catheter mounting comfortable for the patient as an unstabilized sharp edged catheter is generally a source of discomfort for the patient.

Stabilization of the catheter is generally accomplished by applying a dressing to the patient's skin over the catheter hub and associated tubing in an area near the insertion point. In addition to stabilization, the dressing must accomplish a number of other tasks. For example, the dressing must properly cover the exposed insertion point to prevent potential infections. Accordingly, various dressings have been developed, which accomplish some of the tasks required of the dressing.

However, it would be desirable to have a kit available for a health care provider which includes the accessories necessary for efficiently and effectively mounting the catheter on a patient.

### SUMMARY OF THE INVENTION

The present invention provides a catheter patient mounting accessory kit, which includes accessories adapted for different catheter mounting functions. The kit makes available to a health care provider a number of various types of accessories applicable to close an opening associated with a dressing for a catheter inserted into a patient, cushion an IV connector, and/or secure medical tubing or connectors.

In an exemplary embodiment of the present invention, the catheter patient mounting accessory kit includes a release liner having a support side. The kit also includes at least one accessory member having an adhesive side and an opposite non-adhesive side adapted for use in combination with a medical dressing. Additionally, the kit includes at least one other accessory member having an adhesive side and an opposite non-adhesive side, and being adapted for use as a means for securement. Each of the accessory member adhesive sides are releasably disposed on the release liner support side.

Optionally, the accessory members may have a plurality of geometric shapes. The geometric shapes may include at least one of a circular shape, a rectangular shape, a T-shape, an L-shape, and a bow tie shape. The catheter dressing accessory kit may include at least one other accessory member having an adhesive side and an opposite non-adhesive side adapted for cushioning a medical device.

The release liner may include at least one perforation line disposed between two or more of the accessory members. Additionally, the release liner of the catheter dressing accessory kit may include a peripheral boundary portion on the support side, the boundary portion being free from contact with the at least one accessory member. The release liner support side may be tackless. The tackless support side may include a layer of silicone material.

These and other features and advantages of the invention will be more fully understood from the following detailed description of the invention taken together with the accompanying drawings.

### BRIEF DESCRIPTION OF THE DRAWINGS

In the drawings:

FIG. 1 is a plan view of a catheter patient mounting accessory kit in accordance with the present invention;

FIG. 2 is a cross-sectional view of the catheter patient mounting accessory kit of FIG. 1 taken along the line 2-2 in FIG. 1;

FIG. 3 is a side view of the catheter patient mounting accessory kit of FIG. 1 folded in half to form a protective guard for a folded dressing, illustrating the dressing partially inserted into the protective guard; and

FIG. 4 is a plan view of an alternative embodiment of a catheter patient mounting accessory kit in accordance with the present invention.

### DETAILED DESCRIPTION OF THE INVENTION

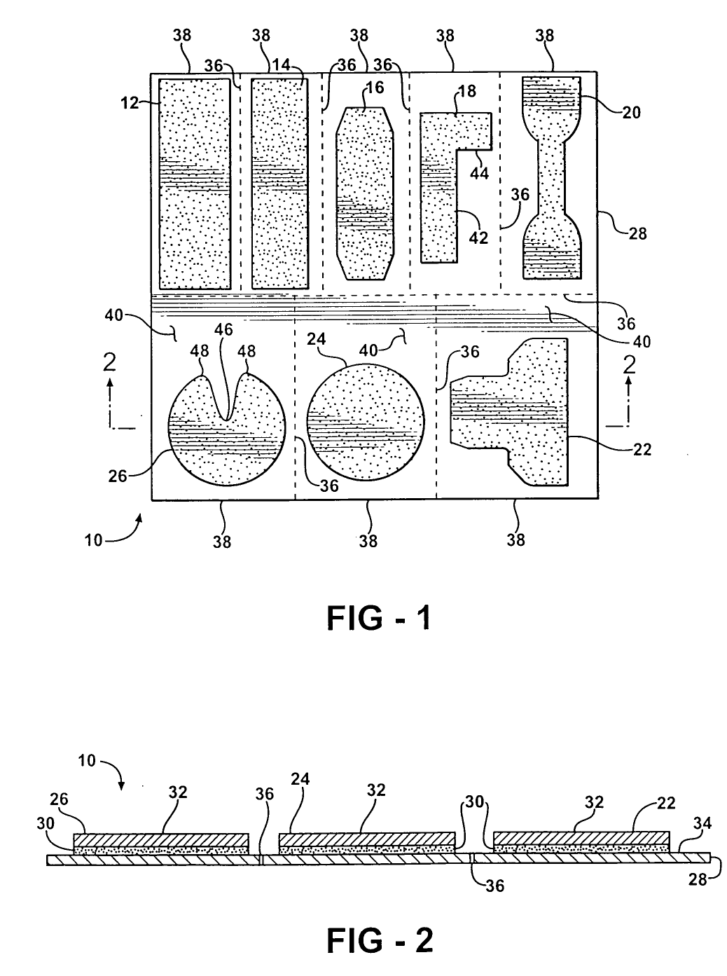

Referring to FIGS. 1 and 2, numeral 10 indicates an embodiment of a catheter patient mounting accessory kit in accordance with the present invention. The accessory kit 10 includes a plurality of accessory members 12, 14, 16, 18, 20, 22, 24 and 26 (i.e. 12-26) releasably disposed on a release liner 28.

The accessory members 12-26 are adapted to be used in connection with mounting a catheter to a patient and may be used to secure a catheter assembly (not shown) to a patient. The catheter assembly components generally include a catheter coupled to a front end of an IV connector, and IV tubing extending out of an opposing rear end of the connector. The catheter includes a sheath for insertion into the patient and a hub for coupling to the IV connector. The medical dressing is applied to the patient's skin over one or more of the components of the catheter assembly.

The accessory members 12-26 may be used in combination with the dressing or any of the components of the catheter assembly. This includes, but is not limited to, using the accessory members 12-26 for various specialized tasks over, under, and/or around the hub, IV connector and/or IV tubing. Generally, cushions typically go under hubs, connectors, and/or tubing while strips typically go over hubs, connectors, and/or tubing.

The accessory members 12-26 each include a thin piece of gas permeable foam-like material having an adhesive side 30 and an opposite non-adhesive side 32. The adhesive side 30 of each accessory member 12-26 is disposed on a tackless support side 34 of the release liner 28. The support side 34 is made tackless via a thin layer of silicone material, which enables the accessory members 12-26 to be selectively removed from the release liner 28 without damage. The accessory members 12-26 may be spacedly disposed on the support side 34.

Though the accessory members 12-26 are described as being composed of gas permeable foam-like material, they may alternatively be made of any appropriately gas permeable resilient material such as rubber, plastic or the like. Additionally, even though the support side 34 is made tackless via silicone, other appropriate materials may be utilized to accomplish the same result.

The release liner 28 may include a plurality of perforation lines 36 disposed between two or more of the accessory members 12-26. The perforation lines 36 define separable portions 38 of the release liner 28, each having a single accessory member 12-26 disposed thereon. The separable portions 38 may be conveniently torn at the perforation lines 36 to separate each single accessory member 12-26 from the rest of the accessory kit 10.

Each separable portion 38 of the liner 28 may include a peripheral boundary portion 40, which is free from contact with its associated accessory member 12-26. The boundary portions 40 are sized to be readily grasped by a gloved hand so that a health care provider may easily lift the associated accessory member 12-26 off the liner's support surface 34.

The accessory members 12-26 may have a plurality of geometric shapes, which are adapted to perform various specific functions in combination with a medical dressing (not shown) applied to a patient's skin over a catheter assembly. For example, accessory members 12, 14 and 16 are generally rectangular shaped to optionally tape and secure the IV tubing exiting the catheter assembly to various anchoring objects such as a patient's limb, a bedrail, or the like.

Accessory member 18 is generally L shaped and includes a main cushion leg 42 and a tab leg 44. The tab leg 44 may be easily grasped by a health care provider, so that the main leg 42 can be conveniently slid under the hub or straight connector of an inserted catheter transverse to the main axis of the connector, hub, or fitting in order to provide cushioning between the hub and patient without lifting the apparatus much.

Accessory 20 is bow tie shaped and is designed to be taped to a patient's skin over a catheter hub. The accessory 22 is T shaped, and is specifically designed to provide cushioning to a generally right-angled IV connector or a tee-connector. Accessory 24 is circularly shaped and may be utilized as an ID label, which can be optionally applied to the dressing and/or skin of a patient to close a dressing opening.

Accessory 26 includes a generally pie shaped cut out and is designed to close or seal the air gaps of a dressing applied to a catheter assembly. That is, a dressing is generally applied to a patient's skin over the IV tubing exiting a catheter assembly. The dressing may be tautly stretched over the rounded IV tubing as it is taped to the patient's skin, forming small tent shaped air gaps on either side of the IV tubing. Airborne contaminants could potentially enter the catheter's access site through these air gaps. In order to seal the air gaps, the wedge section 46 of accessory 26 is slid under the IV tubing up to the dressing. Once in place, the two apex sections 48 of accessory 26 straddle the IV tubing. The apex sections 48 are then lifted up and over the portion of the dressing about the tubing, which forms the entrance to the air gaps, closing the gaps off.

In an alternative embodiment, one or more of the accessory members may include adhesive on both sides. In other words, the non-adhesive side 32 would instead be a second adhesive side opposite the first adhesive side 30. In this embodiment, the accessory kit also may include a second, separate release liner releasably mounted on the second adhesive side of each accessory member having a second adhesive side. Each second release liner may be sized to be at least large enough the cover one of the second adhesive sides of one of the accessory members. “Double-sided” adhesive accessory members have the advantage of being capable of both cushioning and mounting a medical device such as a catheter. For example, one adhesive side of the double-sided adhesive accessory member may be mounted to a catheter while the other side may be mounted to a patient's skin. The catheter is thereby tentatively and preliminarily secured, and a nurse or other medical clinician would no longer have to hold the catheter prior to dressing the catheter.

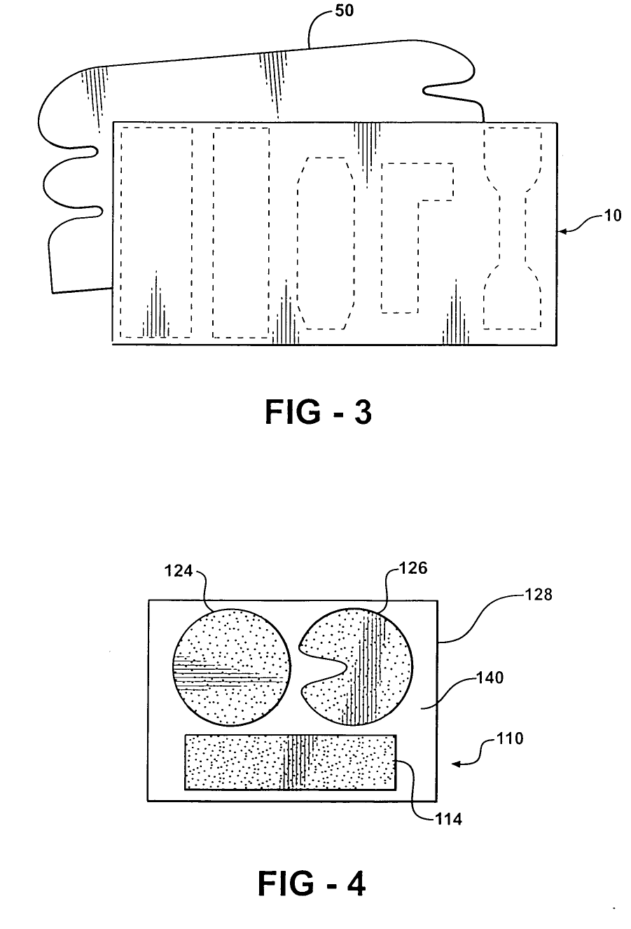

Referring to FIG. 3, the accessory kit 10 may be included in larger catheter related kits as well. For example, accessory kit 10 may be folded in half along one of its perforated lines 36 to provide a protective guard or wallet for a typical IV dressing 50 that is folded in half prior to use. As illustrated in FIG. 3, the IV dressing 50 may be inserted between the folds of the accessory kit 10, which may then in turn be packaged with an IV catheter kit (not shown).

Referring to FIG. 4, in an alternative embodiment of the present invention, a catheter patient mounting accessory kit 110 includes accessory members 114, 124, and 126, which are similar to accessory members 14, 24, and 26 present in the first embodiment 10. The accessory members 114, 124, 126 are releasably disposed on a release liner 128. The release liner 128 includes a peripheral boundary portion 140 that is free from contact with the accessory members 114, 124, and 126. In this particular kit 110, the accessory members 114, 124, and 126 are generally used in combination with IV tubing exiting a dressing that covers and protects a catheter inserted into a patient. That is, accessory member 126 is used to provide closure to the gaps formed around the IV tubing exiting the dressing, accessory member 114 can be used to secure the IV tubing upstream of the dressing, and accessory member 124 enables the optional use of labels in combination with the medical dressing as well as the covering of an opening in the medical dressing.

The present invention provides a kit 10 including an assortment of accessories providing different catheter mounting functions. This enables a health care provider to select an appropriate accessory at the time of application. Further, catheter dressing accessory kits 10 and 110 enable time efficient application of the various accessory members 12-26 without the provider unnecessarily hunting for loose accessory members. Also, the kits offer manufacturing and handling economies and cost effectiveness compared to multiple loose pieces.

Although the invention has been described by reference to specific embodiments, it should be understood that numerous changes may be made within the spirit and scope of the inventive concepts described. Accordingly, it is intended that the invention not be limited to the described embodiments, but that it have the full scope defined by the language of the following claims.

What is claimed is:

1. A catheter patient mounting accessory kit comprising:
a release liner having a support side;
at least one accessory member having an adhesive side and an opposite non-adhesive side adapted for use in combination with a medical dressing; and
at least one other accessory member having an adhesive side and an opposite non-adhesive side, and being adapted for use as a means for securement;
each of said accessory member adhesive sides being releasably disposed on said release liner support side.

2. The catheter patient mounting accessory kit of claim 1, wherein said accessory members are spacedly disposed on said release liner support side.

3. The catheter patient mounting accessory kit of claim 1, wherein said accessory members have a plurality of geometric shapes.

4. The catheter patient mounting accessory kit of claim 3, wherein said geometric shapes include at least one of a circular shape, a rectangular shape, a T-shape, an L-shape, and a bow tie shape.

5. The catheter patient mounting accessory kit of claim 1, including at least one other accessory member having an adhesive side and an opposite non-adhesive side adapted for cushioning a medical device.

6. The catheter patient mounting accessory kit of claim 1, wherein said release liner includes at least one perforation line disposed between two or more of said accessory members.

7. The catheter patient mounting accessory kit of claim 1, wherein said release liner includes a peripheral boundary portion on said support side, said boundary portion being free from contact with said at least one accessory member.

8. The catheter patient mounting accessory kit of claim 1, wherein said release liner support side is tackless.

9. The catheter patient mounting accessory kit of claim 8, wherein said tackless support side includes a layer of silicone material.

10. The catheter patient mounting accessory kit of claim 1, wherein each of said accessory members is a thin piece of foam-like material.

11. The catheter patient mounting accessory kit of claim 1, wherein:
at least one accessory member is adapted for use as a label;
at least one other accessory member is adapted for use as a means for securing IV tubing; and
at least one other accessory is adapted for use as a means for closing gaps formed between a medical dressing and IV tubing exiting from underneath said dressing.

12. The catheter patient mounting accessory kit of claim 1, wherein said accessory kit is foldable along a perforation line to form a protective guard for a medical dressing.
